(19) United States
(12) Patent Application Publication
(10) Pub. No.: US 2022/0395632 A1
(43) Pub. Date: Dec. 15, 2022

(54) Packaging Assembly

(71) Applicant: Sanofi, Paris (FR)

(72) Inventor: Hardy Kietzmann, Frankfurt am Main (DE)

(21) Appl. No.: 17/772,560

(22) PCT Filed: Oct. 26, 2020
(86) PCT No.: PCT/EP2020/080032

§ 371 Date: Apr. 28, 2022

(30) Foreign Application Priority Data
Nov. 1, 2019 (EP) 19306421.9

(51) Int. Cl. A61M 5/00; A61M 5/32

(52) U.S. Cl. CPC A61M 5/002; A61M 5/3202

(57) ABSTRACT

A packaging assembly comprises a case configured to at least partially contain a plurality of injection devices for delivering a medicament, the case being formed as a box having at least one open end face; a lid coupled to the case and movable between an open position and a closed position, the lid being configured to hinge at an edge of the lid furthest from the at least one open end face of the case on another face of the case, and being arranged in the closed position to extend substantially over the open end face of the case and at least partially over another second face of the case that is adjacent to the open end face; and a handle portion having an upper side and a lower side and being arranged to protrude from the lid.

### CROSS REFERENCE TO RELATED APPLICATIONS

The present application is the national stage entry of International Patent Application No. PCT/EP2020/080032, filed on Oct. 26, 2020, and claims priority to Application No. EP 19306421.1, filed on Nov. 1, 2019, the disclosures of which are incorporated herein by reference.

### TECHNICAL FIELD

The application relates to a packaging assembly for a medicament and, in particular, although not exclusively, to a packaging assembly configured to provide a reminder alert at a scheduled dosing time.

### BACKGROUND

Patients suffering chronic disease require regular treatment with medicaments, e.g. on the basis of a predefined schedule. Particular medicaments require refrigerated storage, and are often stored refrigerated in a household refrigerator or fridge. In a home treatment environment, the patient stores the medicament in their fridge and administers a predefined dose as required. Hence, the medicament is typically provided in a secondary packaging for convenient placement and storage in the household fridge. However, the medicament must be stored together with other items that require constant refrigeration, such as foodstuffs and beverages.

Depending on the dosage form of the medicament, the secondary packaging containing the medicament may store a primary packed medicament itself, or may store one or more different kinds of drug delivery devices. For instance, the medicament may be provided in a pre-filled syringe or pen-type injector.

A chronic disease suffered by a patient may cause a loss of mobility, strength and dexterity in the hands and fingers. For example, rheumatoid arthritis is an inflammatory systemic disease of the connective tissue, mainly affecting the joints. Sufferers of rheumatoid arthritis may experience swelling on the fingers and middle joints of the hands, morning stiffness of the fingers and wrists, and pressure pain of the finger rear or toe-rear joints in lateral compression. Patients may be unable to form a closed first or a pinch grip, and may also have reduced strength in the hands and fingers.

### SUMMARY

Embodiments of the present disclosure provide a packaging assembly including a case configured to at least partially contain a plurality of injection devices for delivering a medicament, the case being formed as a box having at least one open end face; a lid coupled to the case and movable between an open position and a closed position, the lid being configured to hinge at an edge of the lid furthest from the open end face of the case on the one other face, and being arranged in the closed position to extend substantially over the open end face of the case and at least partially over one other face adjacent to the open end face; and a handle portion having an upper side and a lower side, and arranged to protrude from the lid such that pressure applied to the upper side when the lid is in the closed position causes the lid to move to the open position, and pressure applied to the lower side when the lid is in the open position causes the lid to move to the closed position.

The handle portion may be formed to extend longitudinally along the lid in parallel with a boundary formed between a first portion of the lid extending over the open end face of the case and a second portion of the lid extending partially over the one other face of the case.

The packaging assembly may include one or more spring ejectors configured to urge at least one of the plurality of injection devices at least partially out of the case when the lid is in the open position.

The case may be configured to contain the plurality of injection devices in an arrangement wherein each injection device lies perpendicular to the open end face of the case.

The case may include one spring ejector for each of the plurality of injections devices, each spring ejector being configured to urge at least a portion of the corresponding injection device through the open end face of the case.

The case may be configured to contain the plurality of injection devices in an arrangement wherein each injection device lies parallel to the open end face of the case.

The case may include one spring ejector configured to urge the plurality of injection devices toward to the open end face of the case.

The packaging assembly may include a retaining lip and a release opening.

The plurality of injection devices are urged against the retaining lip by the spring ejector and the retain lip arranged to retain the plurality of injection devices within the case; and

The release slot may be arranged such that, through the release opening, one of the plurality of injection devices can be lifted over the retaining lip.

The lid may comprise a dispense slot configured to contain one injection device.

The spring ejector may be configured to urge the plurality of injection devices towards the lid, such that when the lid is moved from the closed position to the open position, one of the plurality of injection devices is contained in the dispense slot.

The case may be configured to contain the plurality of injection devices in an arrangement wherein each injection device lies perpendicular to the open end face of the case.

The case may include a plurality of flexible bands, each arranged to extend around an end of a corresponding injection device furthest from the open end face of the case, such that the corresponding injection device is urged towards the open end face of the case by the flexible band when the flexible band is pulled tight.

The packaging assembly may include the plurality of injection devices.

Each of the plurality of injection devices may include a loop element formed at one end of the injection device, and the case is configured to contain the plurality of injection devices in an arrangement wherein the loop element of each injection device is adjacent to the open end face of the case.

Other aspects of the present disclosure provide a packaging assembly including a case configured to at least partially contain a plurality of injection devices for delivering a medicament, the case formed as a box having at least one opening; and at least one tray configured to contain at least one of the plurality of injection devices and arranged to slide out of the case through the opening.

The packaging assembly may include one tray for each of the plurality of injection devices.

Each of the plurality of trays may be configured to rotate around its length such that the injection device contained in the tray falls out of the tray.

The packaging assembly may include one tray configured to contain all of the plurality of injection devices.

The tray may be configured, when pulled fully out of the case, to hinge around an edge of the tray which is adjacent to the case.

The tray may be configured to contain the plurality of injection devices in an arrangement wherein each injection device lies perpendicular to the direction in which the tray is configured to slide, and a side panel of the tray may include at least one opening for pushing one or more of the injection devices out of the tray.

The tray may be formed having at least one sloped portion arranged to lift one or more of the injections devices out of the tray when pushed through the at least one opening in the side panel.

The tray may be configured to contain the plurality of injection devices in an arrangement wherein each injection device lies parallel to the direction in which the tray is configured to slide, and a bottom panel of the tray may include at least one opening for pushing one or more of the injection devices upwards out of the tray.

### BRIEF DESCRIPTION OF THE FIGURES

Embodiments of the present disclosure will now be described, by way of example only, with reference to the accompanying drawings, in which:

FIG. 1 is an isometric view of a packaging assembly according to a first exemplary embodiment;

FIG. 2 is an isometric view of the packaging of FIG. 1;

FIG. 3 is an exploded view of the packaging assembly of FIG. 1;

FIG. 4 is a block diagram of an electronics system of the packaging assembly, according to an exemplary embodiment;

FIGS. 5A and 5B are isometric views of a packaging assembly according to an exemplary embodiment;

FIGS. 6A and 6B are isometric views of a packaging assembly according to an exemplary embodiment;

FIGS. 7A and 7B are isometric views of a packaging assembly according to an exemplary embodiment;

FIGS. 8A and 8B are isometric views of a packaging assembly according to an exemplary embodiment;

FIGS. 9A and 9B are isometric views of a packaging assembly according to an exemplary embodiment;

FIGS. 10A and 10B are isometric views of a packaging assembly according to an exemplary embodiment;

FIGS. 11A and 11B are side-on views of an auto-injection device for use with the packaging assembly, according to an exemplary embodiment.

### DETAILED DESCRIPTION

Embodiments of the present disclosure provide a packaging assembly configured to contain and store a plurality of injection devices for delivering a medicament. An injection device is an example of a drug delivery device and may be a pen-injector or an auto-injector. The packaging assembly is configured to provide access to the plurality of injection devices for a patient experiencing reduced mobility, strength and/or dexterity. The packaging assembly may further allow for an easy removal or insertion of injection devices by the patient. The packaging assembly provides a predictable, convenient and independent experience for the patient.

The packaging assembly may include a lid, for safe and discreet storage of the injection devices. The lid may be configured in such a way that the lid can be opened by the patient and the plurality of injections device can be removed, without requiring a closed first or pinch grip.

A drug delivery device, as described herein, may be configured to inject a medicament into a patient. For example, delivery could be sub-cutaneous, intra-muscular, or intravenous. Such an injection device could be operated by a patient or care-giver, such as a nurse or physician, and can include various types of safety syringe, pen-injector, or auto-injector. The injection device can include a cartridge-based system that requires piercing a sealed ampule before use. Volumes of medicament delivered with these various injection devices can range from about 0.2 ml to about 3 ml.

In combination with a specific medicament, the presently described injection devices may also be customized in order to operate within required specifications. For example, the device may be customized to inject a medicament within a certain time period (e.g., about 3 to about 20 seconds for auto-injectors). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, an injection device will often include a hollow needle ranging from about 25 to about 31 Gauge in size. Common sizes are 27 and 29 Gauge.

The injection devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may be activated via an activation mechanism. Such an activation mechanism can include one or more of a button, a lever, a needle sleeve, or other activation component. Activation may be a one-step or multi-step process. That is, a user may need to activate one or more activation mechanism in order to cause the automated function. For example, a user may depress a needle sleeve against their body in order to cause injection of a medicament. In other devices, a user may be required to depress a button and retract a needle shield in order to cause injection.

In addition, such activation may activate one or more mechanisms. For example, an activation sequence may activate at least two of needle insertion, medicament injection, and needle retraction. Some devices may also require a specific sequence of steps to cause the one or more automated functions to occur. Other devices may operate with sequence independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

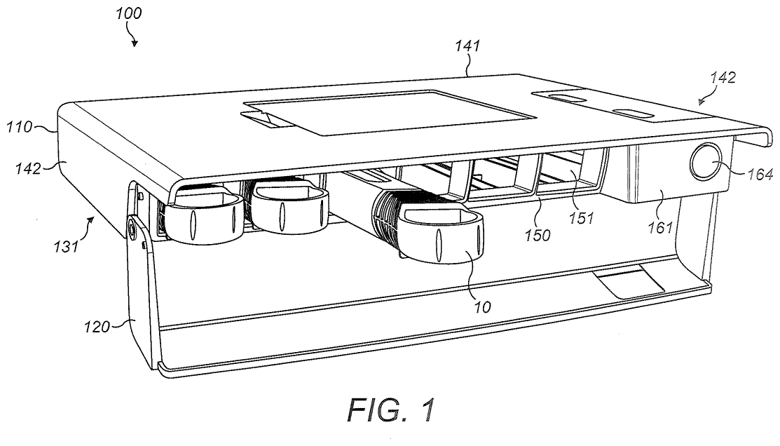

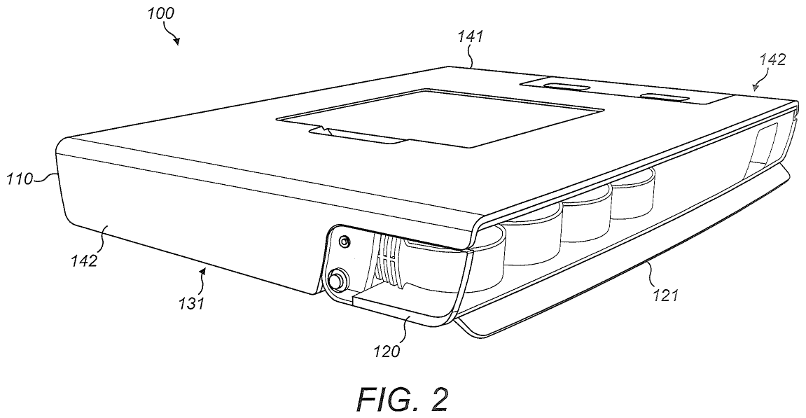

With reference to FIGS. 1 and 2, a packaging assembly 100 according to exemplary embodiments is shown. The packaging assembly 100 comprises a case 110 having a lid 120. The case 110 comprises a bottom face 131, a top face 141, and two side walls 142. The bottom face 131 is curved so as to meet the top face 141 at a rear of the device. At a front end of the case 110, an opening is formed between bottom face 131, the top face 141 and the two side walls 142.

The lid 120 of the case 110 is arranged to cover the opening of the case 110. The lid 120 is attached between the two side walls 142 of the case 110 in a hinged manner. The lid 120 can be freely moved in a hinged manner between a closed position and an open position. In the closed position, the lid 120 is arranged to cover the opening of the case 110. In the open position, the opening of the case 110 is uncovered and an interior of the case 110 can be accessed.

As shown in FIG. 1, the bottom face 131 of the case 110 is shorter than the top face 141. The lid 120 extends from a front edge of the bottom face 131 to a front edge of the top face 141. The lid 120 covers the full extent of a front side of the packaging assembly 100, and a partial extent of a lower side of the packing assembly 100. The lid 120 may be curved. The curve allows the lid 120 to form the entire front and a portion of the bottom of the case 110 in the closed position. Other lid configurations are also contemplated.

The lid 120 may comprise a latching mechanism to hold the lid 120 in the closed position. The latching mechanism may comprise a protruding part arranged at an edge of the lid 120. The protruding part may be configured to engage with a corresponding feature in the case 110 when the lid is in the closed position. The protruding part may be flexible or retractable to disengage from the case 110 and allow the lid 120 to move to the open position.

The lid 120 further comprises a handle 121. The handle 121 is formed to protrude from an outer face of the lid 120. The handle 121 protrudes from the front of the case 110 when the lid 120 is in the closed position. The handle 121 is formed to extend laterally along a width of the case 110, parallel to the top face 141 and the bottom face 131. The handle 121 is formed to extend over at least a portion of the width of the case 110 and may be formed to extend over the full width, between the two side walls 142.

The case 110 is configured to hold and store a plurality of injection devices 10. A depth of the case 110, measured between the rear and the lid 120, is sufficient to accommodate the length of each of the injection devices 10. The depth of the case 110 may be between 160 mm and 180 mm. A height of the case 110, measured between the bottom face 131 and the top face 141, is sufficient to accommodate the width of each of the injection devices 10. The height of the case may be between 30 mm and 40 mm. A width of the case 110, measured between the two side walls 142, is sufficient to accommodate six injection devices 10. The width of the case may be between 180 mm and 200 mm. In some examples, the case may be 188.7 mm wide, 174.7 mm high and 34 mm deep.

The length of the top face 141 may be the full depth of the case 110. The length of the top face 141 may be between 160 mm and 180 mm. The length of the bottom face 131 may be less than the full depth of the case 110. The length of the bottom face 131 may be between 130 mm and 150 mm. A depth of overhang of the top face 141 beyond the front of the bottom face 131 may be between 30 mm and 40 mm. A portion of the length of each injection device 10 may be exposed, corresponding to the depth of the overhang.

The bottom face 131, the top face 141 and the two side walls 142 are formed from an opaque material, for example, an opaque plastic material. The lid 120 is formed from a translucent or frosted material, for example, a clear plastic material with a frosted coating or a treated surface. A portion of the lid 120 may be clear and transparent to form a viewing window through the lid 120.

The case 110 further comprises an internal structure 150 arranged within the opening. The internal structure 150 is visible only when the lid 120 of the case 110 is in an open position. When the lid 120 is in the closed position, the lid obscures the internal structure 150 from view. The internal structure 150 comprises a plurality of openings 151. The openings 151 are configured to hold a corresponding plurality of injection devices 10. The internal structure 150 comprises a row of six openings 151, so as to hold six injection devices 10 arranged in a row along a width of the case 110. The injection devices 10 are held in a longitudinal arrangement, extending along the depth of the case 110. The packaging assembly 100 may be configured to hold more than six, or fewer than six injection devices 10 in the case 110.

The openings 151 are generally rectangular in profile. The openings 151 may be square shaped, or circular shaped to accommodate other sizes of injection device 10. The width of each opening is sufficient to accommodate the width of each injection device 10. Each of the openings 151 opens into an interior space (not shown) for retaining the injection device 10. The internal structure 150 is formed such that each of the openings 151 exposes a portion of the injection device retained within the opening 151. The openings 151 are formed to expose an end portion of each injection device 10 on the lower side of the case 110.

The lid 120 may be configured to retain the plurality of injection devices 10 in position within the case 110 when in the closed position. The lid 120 may be arranged in the closed position to prevent the injection devices 10 from falling or sliding out of the case 110. Each injection device 10 may be retained in position within the corresponding opening 151 by a friction fit with the opening 151.

A retention mechanism may retain the plurality of injection devices 10 in position within the openings 151. The retention mechanism may comprise a mechanical catch configured to engage with each injection device 10, for example, a sprung push-catch push-release mechanism. The injection device 10 is pushed into the opening 151 and pushed against a spring of the retention mechanism to engage a catch. The injection device 10 is pushed a second time to release the catch. A release button or switch may be provided for each of the openings 151, which is configured to release the catch of the retention mechanism when pressed.

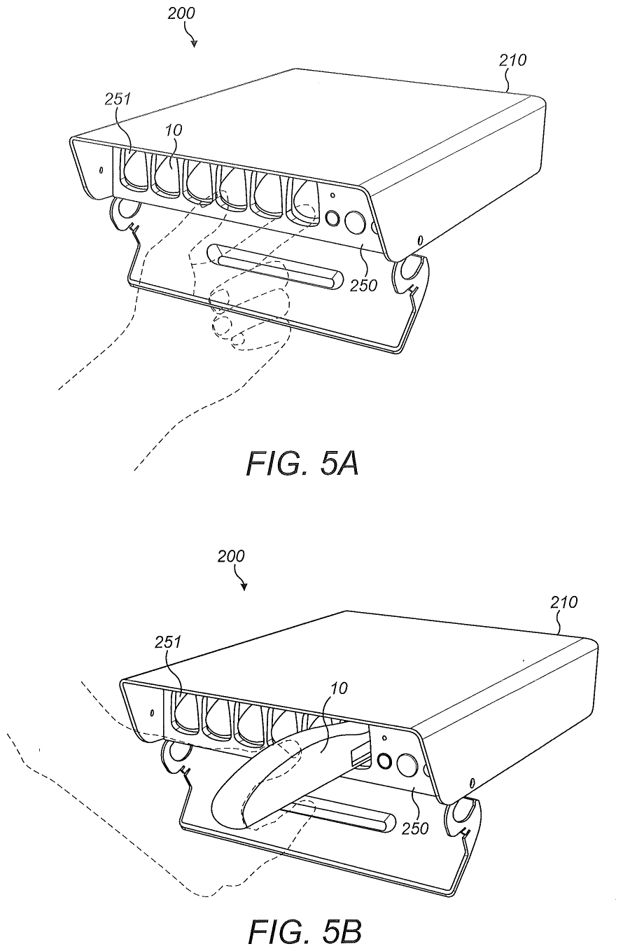

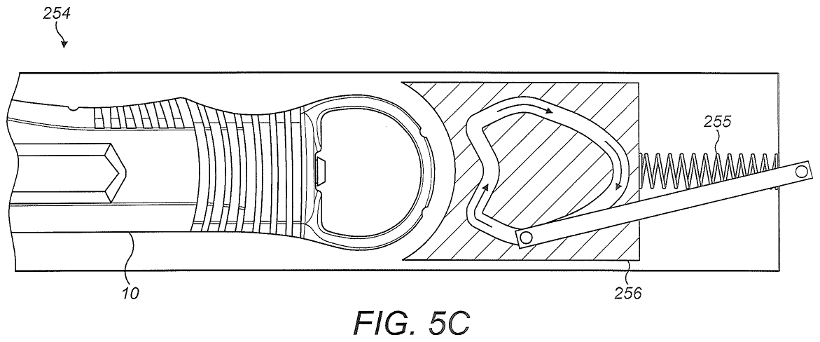

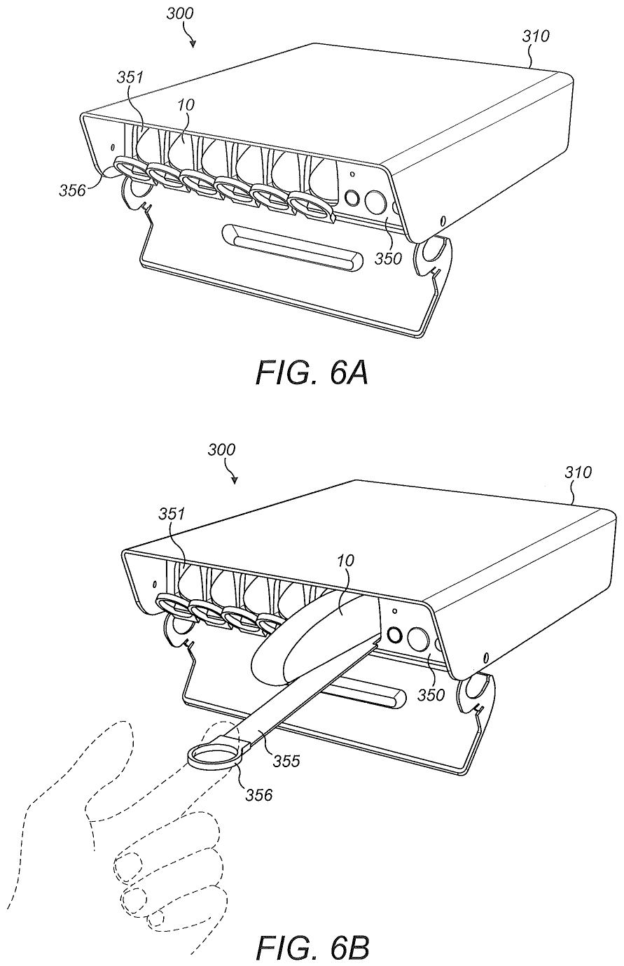

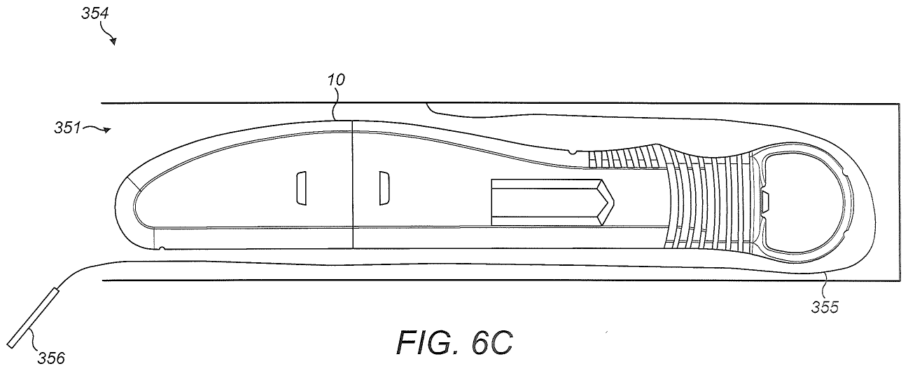

An ejection mechanism may be configured to push one or more of the injection device 10 out of the case 110. The ejection mechanism may comprise one or more springs arranged to push each injection device 10 out of the case 110. Alternatively, a flexible band may be provided for each injection device 10, to pull the injection device out of the case 110. The ejection mechanism will be described in more detail bellow with respect to FIG. 5 and FIG. 6.

FIG. 2 shows the packaging assembly 100 with the lid 120 in the closed position. The handle 121 extends laterally along a width of the case 110. The handle 121 extends over a portion of the width of the case 110 which is less than the full width of the case 110. A width of the handle 121 may be between 80 mm and 110 mm. The handle 121 protrudes from the front of the case 110 when the lid 120 is in the closed position. A depth of the handle 121 may be between 5 mm and 20 mm. A thickness of the handle 121 may be between 2 mm and 10 mm. The handle 121 may be formed to extend at angle below the horizontal when the lid 120 is in the closed position. The handle 121 may be angled at between 10° and 20° when the lid 120 is in the closed position.

The handle 121 is formed having an upper face and a lower face. The handle 121 is formed to move the lid 120 between the open and closed positions by the application of pressure. Downward pressure on the upper face when the lid 120 is in the closed position will cause the lid to move to the open position. Upward pressure on the lower face when the lid 120 is in the open position will cause the lid to move to the closed position. In this case, the interior of the case 110 can be accessed and closed by the simple application of direct pressure, without the need for a pinch grip or other fine motor skills.

A user may receive the packaging assembly 100 in an empty condition. When the user is supplied with a plurality of injection devices 10 they can be loaded into the packaging assembly 100. The lid 120 is moved into the open position and each of the injection devices 10 is inserted into a corresponding one of the openings 151. The lid 120 is moved into the closed position. The packaging assembly 100 is placed in the fridge until a scheduled dosing time is due.

For example, a dosing time may be scheduled every 14 days or 28 days, according to the form of medicament provided in the plurality of injection devices 10. In some embodiments, a period of time between scheduled dosing times may be between 2 days and 60 days, according to the requirements of the medicament.

The packaging assembly 100 may be configured to provide the user with a visual and/or reminder when the scheduled dosing time is due. The packaging assembly 100 may be further configured to determine whether the lid 120 is in the open position or the closed position, and may deactivate the reminder upon detection of the lid 120 being moved to the open position. When the reminder is active, the user can push the handle 121 to move the lid to the open position and the reminder will also be deactivated. In this way, the reminder can be easily deactivated with fewer inputs and without the need for fine motor skills e.g. to move a switch or push a button.

Functionality of the packaging assembly 100 is provided by the following electronics:

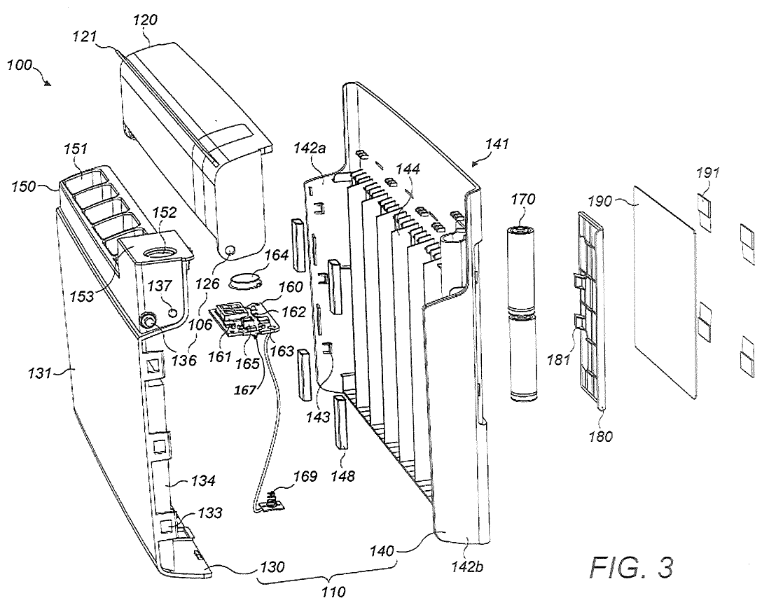

The packaging assembly 100 includes an electronics system 160 (not visible in FIG. 1, but visible in FIG. 3). The electronics system comprises multiple components that are connected together to provide a specific set of functions, described below. The components of the electronics system 160 are mounted on a printed circuit board (PCB), although instead they may be interconnected through some other medium.

The electronics system 160 is attached to the internal structure 150. Some of the electronic components of the electronics system 160 are user interface hardware components and together provide a user interface. The components that provide the user interface are positioned at one end of the row of openings 151 of the internal structure 150.

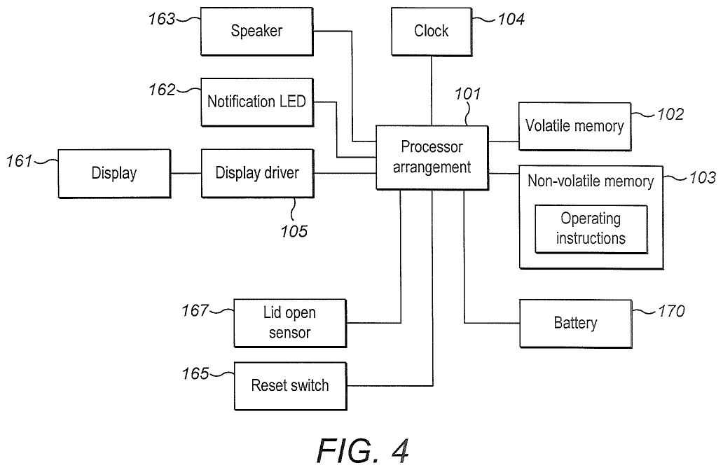

The electronics system 160 is shown schematically in FIG. 4. The electronics system 160 comprises a processor arrangement 101, which is shown in FIG. 4. The processor arrangement 101 controls operation of the other hardware components of the electronics system 160. The processor arrangement 101 is configured to control the hardware components which form the user interface. The processor arrangement 101 is configured to process one or more input signals from at least one input sensor.

The electronics system 160 comprises a display 161. The display 161 is an example of an optical transducer. The display 161 comprises two seven-segment light-emitting diode (LED) arrays. The display 161 may be visible to the user through the transparent or translucent lid 120 or, in some embodiments, through a transparent viewing window in the lid 120. The electronics system 160 comprises a light-emitting diode (LED) 162. The LED 162 is an example of an optical transducer. A colour of the LED 162 is different to a colour of the seven-segment LED arrays in the display 161, for example, the colour of the LED 162 is red and the colour of the display 161 is blue. The electronics system 160 comprises a reset button 164. The reset button 164 is an example of an input device. The reset button 164 is a sprung plunger button which may be depressed by the user. The electronics system 160 comprises a speaker 163 (not shown in this Figure). The speaker 163 is an example of an audio transducer.

With further reference to FIG. 3, an exploded view of the packaging assembly 100 according to the first embodiment is shown. The case 110 of the packaging assembly 100 comprises a first part 130 and a second part 140.

The first part 130 of the case 110 is formed from a single piece. The first part 130 of the case 110 comprises the bottom face 131 and the rear of the packaging assembly 100. Along each side edge of the bottom face 131, a plurality of openings 133 are formed for engaging with the second part 140 of the case 110. Three openings 133 are formed along each edge of the first part 130. The first part 130 further comprises a plurality of dividers 134 for holding the plurality of injection devices 10 (not shown in FIG. 2) in position within the case 110. Each side of the first part 130 comprises a first hinging part 136. Each side of the first part 130 comprises a first latching part 137.

The internal structure 150 is formed from in the first part 130 of the case 110. The internal structure 150 comprises the plurality of openings 151. The openings 151 are configured to hold the corresponding plurality of injection devices 10. The internal structure 150 further comprises one or more openings 152 for the hardware components of the user interface. The openings 152 are arranged in a flat panel 153 of the internal structure 150. The flat panel 153 is arranged adjacent to the openings 151.

The second part 140 comprises the top face 141, a first side wall 142a and a second side wall 142b of the case 110. The length of the first side wall 142a and the second side wall 142b may be less than the full depth of the case 110. The length of the first side wall 142a and the second side wall 142b may be between 130 mm and 150 mm. The second part 140 is formed from a single piece. The second part 140 further comprises a plurality of dividers 144 for holding and storing the plurality of injection devices 10 in position within the case 110. The dividers 144 of the second part 140 are aligned with the dividers 134 of the first part 130.

The case 110 of the packaging assembly 100 comprises a plurality of magnets 148. The magnets 148 are fixed in position on an internal side of the top face 141. The case comprises four magnets 148 fixed in a square arrangement. The plurality of magnets 148 allows the top face 141 of the case 110 to be releasably attached to a magnetic surface, for example, a steel surface. The magnets 148 may be neodymium magnets.

The packaging assembly 100 further comprises a mounting plate 190. The mounting plate 190 comprises a plurality of adhesive strips 191. The mounting plate 190 can be fixed to a surface using the adhesive strips 191, such as, for example, a wall or under a shelf within a fridge. The mounting plate 190 is formed from a magnetic material, for example, steel. The case 110 can be releasable attached to the surface by magnetically attaching to the mounting plate 190.

The mounting plate 190 comprises three adhesive strips 191. The adhesive strips 191 are arranged in parallel across the width of the mounting plate 190 and each adhesive strip 191 extends along substantially the full length of the mounting plate. Alternatively, the mounting plate 190 may comprise only two adhesive strips 191 which are spaced apart on the mounting plate 190, or may comprise more than three adhesive strips 191 extending in parallel. Further alternatively, the mounting plate 190 may comprise four adhesive strips 191 positioned in a rectangular arrangement, for example, at each corner of the mounting plate 190. The mounting plate may comprise any number of adhesive strips 191 arranged in a regular array.

The mounting plate 190 may alternatively be placed, without adhesive, on an upper side of a shelf. The case 110 may be magnetically held beneath the shelf through a magnetic attraction to the mounting plate 190.

The packaging assembly 100 further comprises a plurality of batteries 170. The batteries 170 are arranged to provide power to the components of the user interface. The second part 140 of the case 110 comprises a battery opening 145 formed in the top face 141. The battery opening 145 is configured to receive the plurality of batteries 170. A battery cover 180 is configured to slidably engage with the battery opening 145 of the second part 140 and to cover the battery opening 145 when the packaging assembly 100 is in use. The battery cover 180 comprises a plurality of latches 181 arranged to engage with the second part 140 of the case 110.

Each of the first side wall 142a and the second side wall 142b of the case 110 comprises a plurality of engaging hooks 143. The engaging hooks 143 are arranged on an inner face of the respective side wall. Each of the side walls 142 comprises three engaging hooks 143. The engaging hooks 143 are each configured to engage with the corresponding opening 133 in the first part 130 of the case 110.

The lid 120 of the case 110 comprises a second hinging part 126 configured to engage with the first hinging part 136 of the first part 130 of the case 110. The first hinging part 136 and the second hinging part 126 together form a hinge 106 for attaching the lid 120 to the first part 130 of the case 110. For example, the second hinging part 126 comprises an opening and the first hinging part 136 comprises a protrusion arranged to fit within the opening of the second hinging part 126. The first hinging part 136 is configured to rotate within the opening of the second hinging part 126.

The lid 120 of the case 110 comprises a second latching part (not shown) configured to engage with the first latching part 137 of the first part 130 of the case 110. The second latching part is configured to releasably engage with the first latching part 137 to maintain the lid 120 in a closed position. For example, the first latching part 137 comprises a protrusion and the second latching part comprises a opening configured to releasably engage with the protrusion of the first latching part 137.

The lid 120 is formed from a translucent plastic material. A portion of the lid 120 may be clear and transparent to form a viewing window through the lid 120. The handle 121 is formed as part of the lid 120 and extends outwards from the body of the case 110.

The packaging assembly 100 comprises the electronics system 160. The electronics system 160 includes the hardware components of the user interface, namely the display 161, the LED 162, the speaker 163 and the reset button 164. The display 161 of the user interface may be visible through the translucent lid 120. The electronics system 160 is coupled with a battery contact 169. The battery contact 169 is mounted with the plurality of batteries 170 in order to supply power to the electronics system 160.

The electronics system 160 comprises a reset switch 165. The reset button 164 is a sprung plunger button arranged to be pushed by the user. The reset switch 165 is a mechanical switch mounted on the electronics system 160. The reset switch 165 is positioned below the reset button 164. The reset switch 165 is arranged to be actuated by the reset button 164. The reset button 164 may be coupled to the reset switch 165.

The electronics system 160 comprises a hinge switch (167). The hinge switch may be an electro-mechanical switch such as a microswitch or other miniature snap action switch. The hinge switch is an example of a lid open sensor.

The hinge switch is arranged to engage with the lid 120 of the case 110 when the lid 120 is in a closed position. An actuating part of the lid 120 may be shaped so as to press the hinge switch 167 when the lid 120 is in a closed position. The hinge switch may be mounted at an edge of the PCB of the electronics system 160. The actuating part of the lid 120 may be arranged to pass the edge of the PCB of the electronics system 160 when the lid 120 is in a closed position.

The electronics system 160 further comprises the processor arrangement 101 (not shown in this Figure). The processor arrangement 101 is configured to process the input signals from the one or more sensors and the switches on the electronics system 160. The processor arrangement 101 is configured to control the outputs of the user interface elements on the electronics system 160.

With respect to FIG. 4, a schematic representation of the electronics system 160 of the packaging assembly 100 according to the first embodiment is shown. The electronics system 160 comprises the processor arrangement 101. The processor arrangement 101 and other hardware components may be connected via a system bus (not shown). Each hardware component may be connected to the system bus either directly or via an interface. A battery 170 is arranged to provide power to the electronics system 160.

The processor arrangement 101 controls operation of the other hardware components of the electronics system 160. The processor arrangement 101 may be an integrated circuit of any kind. The processor arrangement 101 may for instance be a general purpose processor. It may be a single core device or a multiple core device. The processor arrangement 101 may be a central processing unit (CPU) or a general processing unit (GPU). Alternatively, it may be a more specialist unit, for instance a RISC processor or programmable hardware with embedded firmware. Multiple processors may be included. The processor arrangement 101 may be termed processing means.

The processor arrangement 101 has a clock speed of 2 Hz. The clock speed is selected to provide a balance between power usage and usability. A greater clock speed provides improved usability by reducing the time required for the processor arrangement 101 to respond to an input. However, a greater clock speed will increase the power usage of the processor arrangement 101. The clock speed may be selected between 0.5 and 100 Hz.

The electronics system 160 comprises a working or volatile memory 102. The processor arrangement 101 may access the volatile memory 102 in order to process data and may control the storage of data in memory. The volatile memory 102 may be a RAM of any type, for example Static RAM (SRAM), Dynamic RAM (DRAM), or it may be Flash memory. Multiple volatile memories may be included, but are omitted from the Figure.

The electronics system 160 comprises a non-volatile memory 103. The non-volatile memory 103 stores a set of operation instructions for controlling the normal operation of the processor arrangement 101. The non-volatile memory 103 may be a memory of any kind such as a Read Only Memory (ROM), a Flash memory or a magnetic drive memory. Other non-volatile memories may be included, but are omitted from the Figure.

The processor arrangement 101 operates under the control of the operating instructions. The operating instructions may comprise code (i.e. drivers) relating to the hardware components of the electronics system 160, as well as code relating to the basic operation of the packaging apparatus. The operating instructions may also cause activation of one or more software modules stored in the non-volatile memory 103. Generally speaking, the processor arrangement 101 executes one or more instructions of the operating instructions, which are stored permanently or semi-permanently in the non-volatile memory 103, using the volatile memory 102 temporarily to store data generated during execution of the operating instructions.

The processor arrangement 101, the volatile memory 102 and the non-volatile memory 103 may be provided as separate integrated circuit chips connected by an off-chip bus, or they may be provided on a single integrated circuit chip. The processor arrangement 101, the volatile memory 102 and the non-volatile memory 103 may be provided as a microcontroller.

The electronics system 160 comprises a clock 104. The clock 104 may be a clock crystal, for example, a quartz crystal oscillator. The clock 104 may be a separate component to the processor arrangement 101 which is configured to provide a clock signal to the processor arrangement 101. The processor arrangement 101 may be configured to provide a real time clock based on the signal from the clock 104. Alternatively, the clock 104 may be a clock crystal which is provide on a single integrated circuit chip with the processor arrangement 101.

The processor arrangement 101 is configured to perform a countdown operation. The countdown operation monitors a number of days remaining until the scheduled dosing time. The countdown operation is set and activated in response to an input from the reset switch 165. The predetermined time period for the countdown to the next scheduled dosing time is stored in the non-volatile memory with the operating instructions of the processor arrangement 101. The processor arrangement 101 records the number of days to the volatile memory 102 and every 24 hours reduces the recorded number of days by one.

For example, if the predetermined time period until the next scheduled dosing time is due is 14 days, the countdown operation is started from 14 days.

The electronics system 160 may comprise a timer duration switch. The timer duration switch may be configured to select the time period until the next scheduled dosing time. The timer duration switch may be a slide switch having a first position and a second position. The processor arrangement 101 may be configured to set the time period for the countdown to the next scheduled dosing time based on the position of the timer duration switch. The processor arrangement 101 may set the time period to be 14 days if the slide switch is in the first position. The processor arrangement 101 may set the time period to be 28 days if the slide switch is in the second position.

The position of the timer duration switch may be preset as part of a manufacturing process and may not be adjustable by a user. Alternatively, the timer duration switch may be accessible for a user to set the timer period to the next scheduled dosing time. The timer duration switch may be mounted on a top face of the PCB of the electronics system. The timer duration switch may be accessible through the battery opening 145.

Every 24 hours, the number of days recorded to the volatile memory 102 is reduced by one. After 13 days, when 1 day remains until the scheduled dosing time, the processor arrangement 101 may control the electronics system 160 to generate an output to indicate that the scheduled dosing time is near. After 14 days, on the day of the scheduled dosing time, the processor arrangement 101 may control the electronics system 160 to generate an output to indicate that the scheduled dosing time is due. The hardware components of the electronics system 160 which form the user interface may be controlled to indicate that the scheduled dosing time is due.

On one day, for instance the first day, the reduction of the number of days may be provided in less than 24 hours. For instance, it may be achieved in 20 hours or 22 hours. This can help to prevent creep of the alert time to later and later in the day after multiple resets of the countdown timer. Alternatively, when the remaining number of days recorded in the volatile memory is equal to one, the processor arrangement 101 may be configured to reduce the time remaining until the next scheduled dosing time is due. For example, the processor arrangement 101 may be configured to wait only 23 hours before reducing the number of days to zero. In this way, the time of day at which the scheduled dosing time becomes due is one hour earlier than the time at which the reset button 164 was pressed.

The processor arrangement 101 may be configured to perform one or more timing operations. The processor arrangement 101 may start a timing operation from zero and monitor an increasing amount of time. Alternatively, the processor arrangement 101 may start a timing operation from a predetermined time and count down until the timer expires.

The processor arrangement 101 may be configured to check the state of charge of one or more batteries 170 included in the packaging assembly 100. The state of charge is determined to be low if it is below a threshold (which may be built into the design of the packaging arrangement). The state of charge may be determined by measurement of the voltage provided by the battery 170, by monitoring energy use from a full state of charge, or a combination of these two techniques.

The electronics system 160 comprises a lid open sensor. The lid open sensor 167 is configured to provide a signal to the processor arrangement 101 when the lid 120 of the case 110 in a closed position.

The lid open sensor 167 may be a hinge switch, for example an electro-mechanical switch such as a microswitch or other miniature snap action switch. The lid open sensor 167 may be arranged to mechanically engage with the lid 120 when the lid 120 is in a closed position. An actuating part 128 of the lid 120 may be shaped to engage with the lid open sensor 167 when the lid 120 is in a closed position. The lid open sensor 167 may be a normally open switch having an open state and a closed state. The switch may be operated to move from the open state to the closed state when pressed. The switch may be configured to pass a current in a closed state only.

The lid open sensor 167 may be configured to provide a signal to the processor arrangement 101 when the switch is pressed into the closed state by the lid 120. The processor arrangement 101 may be configured to set a variable to indicate whether or not the lid 120 has been opened.

The electronics system 160 comprises a reset switch 165. The reset switch 165 is configured to provide a signal to the processor arrangement 101 when actuated by the reset button 164. The user presses the reset button 164 to indicate that the scheduled dosage has taken place, and to reset the time period for the next scheduled dosing time.

The reset switch 165 may be a mechanical switch mounted on the electronics system 160. The reset switch 165 is arranged to be actuated by the reset button 164. The reset switch 165 may be a normally open switch having an open state and a closed state. The reset switch 165 may be operated to move from the open state to the closed state when pressed. The reset switch 165 may be configured to pass a current in a closed state only. The reset switch 165 may be configured to provide a signal to the processor arrangement 101 when moved to the closed state.

The reset button 164 may be coupled to the reset switch 165. The reset switch 165 may be positioned below the reset button 164. If the reset button 164 is pressed, the reset switch 165 may be moved to the closed state by the reset button 164. The reset switch 165 is configured to provide a signal to the processor arrangement 101 when actuated by the reset button 164. The processor arrangement 101 is configured to reset the countdown operation in response to the signal from the reset switch 165. The time remaining until the scheduled dosing time is due is reset to be 14 days by the processor arrangement 101. In some embodiments, the user must press and hold the reset button 164 for 2 seconds to reset the time period for the next scheduled dosing time. The packaging assembly 11 may filter out short presses of the reset button 164, so as to reduce the occurrence of falsely triggering the reset operation.

The electronics system 160 comprises the display 161 of the user interface. The display 161 can be operated to provide a notification. The display 161 can be operated to provide an indication of a status of the packaging assembly 100. The display 161 is an example of a status indicator. The display 161 can be operated to show information relating to the status of the packaging assembly 100. The display 161 can be operated to show any number from 00 to 99 by illuminating some or all of the LED segments. Certain letters may also be shown by the display 161.

The electronics system 106 may comprise a display driver 105. The display driver 105 may be provided as a separate integrated circuit chip to the processor arrangement 101, which is connected by an off-chip bus. Alternatively, the display driver 105 may be provided on a single integrated circuit chip with the processor arrangement 101. The display driver 105 may be a port expander for individually controlling the segments of a seven-segment LED display.

The processor arrangement 101 can operate the display 161 to show the number of days remaining until the scheduled dosing time is due. The display 161 can be operated to provide a visual reminder output that the scheduled dosing time is due. The display 161 can be operated further to provide a visual reminder output that the scheduled dosing time is near.

If the countdown operation of the processor arrangement 101 is reset, the countdown operation is started from 14 days. The display 161 is operated to show the number “14” to indicate that 14 days remain. Each day, the number of days shown by the display 161 is reduced by one. After 13 days, when 1 day remains until the scheduled dosing time, the display 161 is operated to show “01”. The display 161 is operated to flash or blink to indicate that the scheduled dosing time is near. The display 161 is operated to flash by intermittently showing “01”.

After 14 days, on the day of the scheduled dosing time, the display 161 is operated to show “00”. The display 161 is operated to flash to indicate that the scheduled dosing time is due. The display 161 is operated to flash by intermittently showing “00”. The flash periodicity of the display 161 may be of the order of 0.25 seconds to 2 seconds.

The processor arrangement 101 may check the state of charge of one or more batteries 170 included in the packaging assembly 100. If the state of charge is determined to be low, the display 161 may be operated to show a battery low warning.

The battery low warning shown by the display 161 may be a message comprising an upper case L on the first seven-segment array, and a lower case o on the second seven-segment array. That is, the display 161 may show the message “Lo”. The battery low warning may be shown intermittently by the display 161 under the control of the processor arrangement 101. The display 161 may be operated to show the battery low warning alternately with the number of days remaining until the scheduled dosing time. The periodicity of the intermittent or alternating operation of the display 161 may be of the order of 0.25 seconds to 2 seconds

The electronics system 160 comprises the LED 162 of the user interface. The LED 162 can be operated to provide a notification. The LED 162 can be operated to provide an indication of a status of the packaging assembly 100. The LED 162 is an example of a status indicator.

The processor arrangement 101 can operate the LED 162 to provide a visual reminder that the scheduled dosing time is due. After 14 days, on the day of the scheduled dosing time, the LED 162 is operated to generate an intermittent output light. The LED 162 is operated to flash or blink in the colour red to provide a visual reminder that the scheduled dosing time is due. The flash periodicity of the LED 162 may be of the order of 0.25 seconds to 2 seconds.

The electronics system 160 comprises the speaker 163 of the user interface. The speaker 163 can be operated to output a notification signal. The speaker 162 can be operated to provide an indication of a status of the packaging assembly 100. The speaker 162 is an example of a status indicator.

The processor arrangement 101 operated the speaker 163 to provide an audio reminder that the scheduled dosing time is due. After 14 days, on the day of the scheduled dosing time, the speaker 163 is operated to output an audio reminder that the schedule dosage time is due. The speaker 163 may be operated to output an intermittent tone. The periodicity of the speaker 163 output may be of the order of 0.25 seconds to 2 seconds.

The processor arrangement 101 controls the operation of the speaker 163 according to the signal input by the lid open sensor 167. When the scheduled dosing time is due, the processor arrangement 101 operated the speaker 163 to output an audio indication that the scheduled dosing time is due, as described above. When the lid open sensor 167 provides a signal to the processor arrangement 101 to indicate that the lid 120 of the case 110 is open, the processor arrangement 101 controls the speaker 163 to deactivate the reminder.

The processor arrangement 101 may control the operation of the speaker 163 according to a stored value of a lid flag. When the lid open sensor 167 provides a signal to the processor arrangement 101 to indicate that the lid 120 is open, the processor arrangement 101 may set the lid flag to have a value of 1. The processor arrangement 101 controls the speaker 163 to deactivate the reminder when the stored value of the lid flag is equal to 1. The processor arrangement 101 may reset the lid flag to have a value of 0 when the reset button 164 is pressed.

In this way, the audio reminder output by the speaker 163 is deactivated only when the lid opened by a user. The speaker 163 is deactivated only when the user opens the lid 120 of the case 110 in order to retrieve the injection device 10 for the scheduled dosage. The packaging assembly 100 thereby improves compliance with the scheduled dosage regime.

With respect to FIGS. 5A and 5B, a packaging assembly 200 according to a second embodiment is shown. Elements not described below are substantially the same as those of the first embodiment.

A case 210 of the packaging assembly 200 comprises a flat panel 250 forming an internal structure of the case 210. The flat panel 250 extends across the full width of the case 210. Openings 251 for the injection devices 10 and openings 252 for the user interface are formed in the flat panel 150.

With respect to FIG. 5C, an ejection mechanism 254 according to the second embodiment is shown. The packing assemble 200 may comprise one or more ejection mechanisms 254, and may comprise one ejection mechanism 254 for each injection device 10. The ejection mechanism 254 comprises at least one ejection spring 255 arranged to push the injection device 10 out of the case 210.

The ejection mechanism 254 may be configured to push a portion of each injection device 10 out of the corresponding opening 251. The ejection mechanism 254 may be biased against a retention mechanism to push each injection device when released by the retention mechanism. The retention mechanism and ejection mechanism may be combined in a push-to-engage and push-to-disengage mechanism. The injection device 10 is pushed into the opening 251 and pushed against a spring of the retention mechanism to engage a catch. The injection device 10 is pushed a second time to release the catch and the ejection spring 255 of the ejection mechanism 254 pushes the injection device 10 at least partially out of the case 110. The spring of the retention mechanism and the ejection spring 255 of the ejection mechanism 254 may be a single spring. The ejection mechanism 254 comprises a camming mechanism 256 to regulate the push-to-engage and push-to-disengage mechanism.

With respect to FIGS. 6A and 6B, a packaging assembly 300 according to a third embodiment is shown. Elements not described below are substantially the same as those of the first embodiment.

A case 310 of the packing assembly 300 comprises a flat panel 350, substantially as described with respect to the second embodiment.

With respect to FIG. 6C, an ejection mechanism 354 according to the third embodiment is shown. The ejection mechanism 354 comprises a flexible band 355 for each of the plurality of injection devices 10. Each band 355 is arranged such that the corresponding injection device 10 is pulled out of the case 310 when the band 355 is pulled. A first end of the band 355 is fixed in position within the case 310. A second end of the band 355 is unattached. The second end is passed through a corresponding opening 151 and remains external to the case 310. A middle section of the band 355 is passed around an end of the injection device 10 within the case 310. In this way, pulling the band 355 will pull the injection device 10 out of the case 310.

A width of the band 355 is less than the width of each opening 151, such that the band 355 can pass through the opening 351. The width of the band 355 the same or nearly the same as the width of each injection device 10, such that the injection device 10 pushes the band 353 into position when the injection device 10 is inserted into the case 310.

The second end of the band 355 comprises a ring 356 for pulling the band 355. An internal diameter of the ring 356 is large enough to pass a finger through the ring 356 and pull the band 355. In this way, it is possible to pull the band 355 without forming a pinch grip. An external diameter of the ring 356 may be larger than the opening 351, such that the second end of the band 355 cannot pass inside the case 310. Alternatively, the second end of the band 355 may be formed with a hook or a t-shaped handle for pulling the band 355.

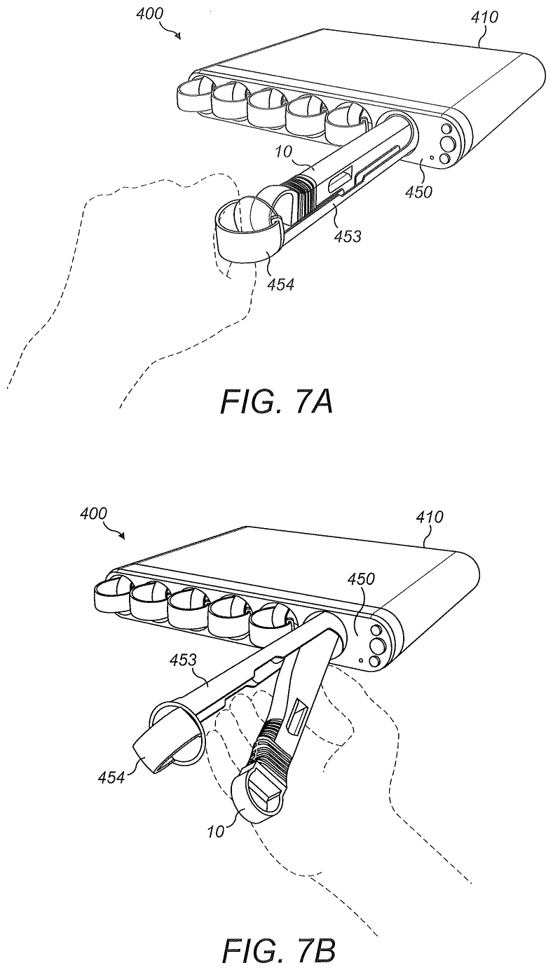

With respect to FIGS. 7A and 7B, a packaging assembly 400 according to a fourth embodiment is shown. Elements not described below are substantially the same as those of the first embodiment.

A case 410 comprises a flat panel 450, substantially as described with respect to the second embodiment.

The case 410 comprises a plurality of trays 453 corresponding to the number of injection devices. Each of the trays 453 is configured to hold one of the injection devices 10. The trays 453 are cylindrical, and open on one side such that an injection device 10 can be dropped into the tray 453 sideways. The case 410 comprises six trays 453 corresponding to each of the openings 451. Each of the trays 453 is arranged to slide into and out of a corresponding opening 451. A retaining lip at one end of the tray 453 may prevent the tray 453 from being fully removed from the case 410. The trays 453 may be rotated within the opening to drop the injection device 10 out of the tray 453. The user can rotate the tray 453 until the open side is facing downward and the injection device 10 falls out of the tray 453. The trays 453 may comprise one or more retaining grooves or rails to maintain the tray in an upright orientation when sliding in and out of the opening 451. The retaining grooves or rails may be configured such that the tray 453 is able to rotate once the tray 453 is withdrawn to its full extent from the case 410.

The outer end of the tray 453 comprises a ring 454 for pulling the band. An internal diameter of the ring 454 is large enough to pass a finger through the ring 454 and pull the tray 453. In this way, it is possible to pull the tray 453 without forming a pinch grip. Alternatively, the outer end of the tray 453 may be formed with a hook or a t-shaped handle for pulling the tray 453.

The retaining mechanism may lock one or more of the trays 453 in position when fully inserted into the case 410. According to an embodiment, the case 410 is formed without a lid. Alternatively, the plurality of trays 453 may be provided in combination with a lid.

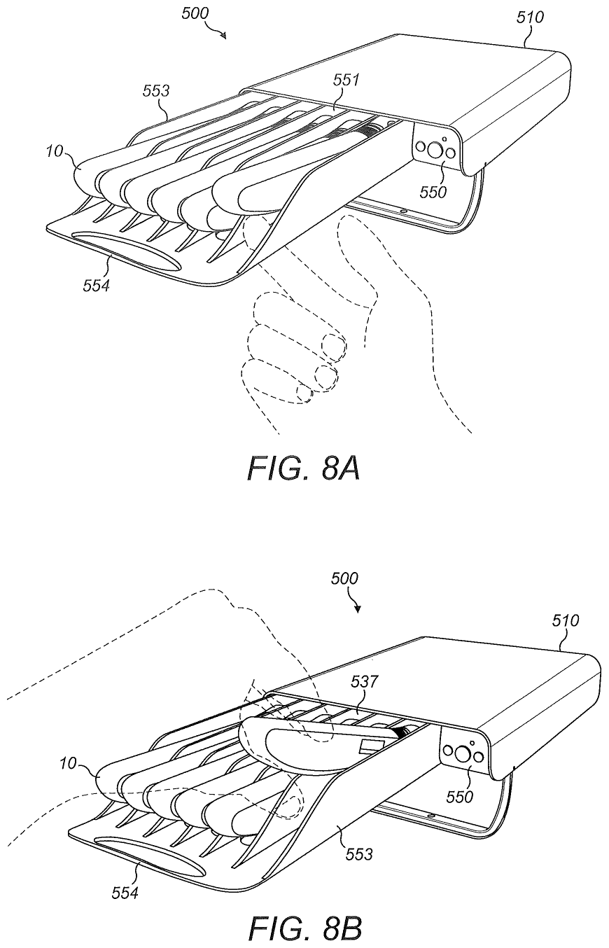

With respect to FIGS. 8A and 8B, a packaging assembly 500 according to a fourth embodiment is shown. Elements not described below are substantially the same as those of the first embodiment.

An internal structure 550 of the case 510 is formed with a single opening 551. The opening 551 is generally rectangular. The opening 551 extends over the full height of the case 510 and across the majority of the width of the case 510, up to the flat panel which holds the user interface.

The case 510 comprises a tray 553 configured to hold the plurality of injection devices 10. The tray 553 is generally rectangular in shape. The tray 553 comprises a base, two side walls and a rear wall (not shown). The tray 553 is open on an upper side and a front end, such that the plurality of injection devices 10 can be dropped into and lifted out of the tray 553. A plurality of dividing walls are arranged to retain the injection devices 10 in position within the tray 553. The injection devices 10 are held in a longitudinal arrangement, extending along the depth of the case 510.

The tray 553 is configured to slide into and out of the opening 551. A retaining element at the rear end of the tray 553 may prevent the tray 553 from being fully removed from the case 510. The tray 553 may be configured to pivot downwards when withdrawn to the full extent from the case 510. The tray 553 may pivot downwards between 10° and 20°, to present the injection devices 10 to the user.

The base of the tray 553 is formed to have a handle 554 at a front end, to pull the tray 553 out of the case 510. The handle 554 may be formed by an opening or, alternatively, by a protrusion to be grasped. The base further comprises a plurality of openings 555, corresponding to the number of injection devices 10. The openings 555 are arranged between the dividing walls, at a front end of the base. A diameter of each opening 555 is large enough for the user to insert a finger, in order to lift the injection device 10 out of the tray 553. In this way, the injection devices 10 can be easily removed from the tray 553 without needing to form a pinch grip.

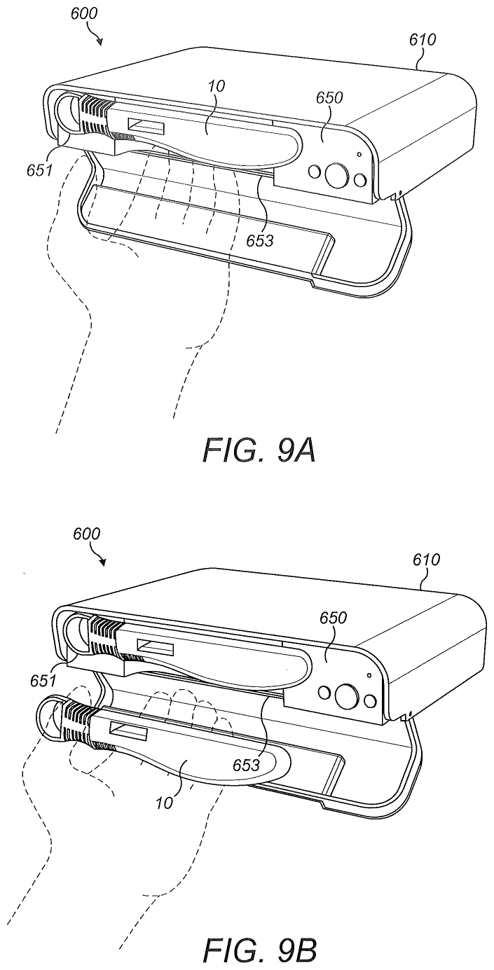

With respect to FIGS. 9A and 9B, a packaging assembly 600 according to a fourth embodiment is shown. Elements not described below are substantially the same as those of the first embodiment.

The internal structure 650 of the case 610 is formed with a single opening 651. The opening 651 is formed such that an injection device 10 can be passed sideways through the opening 651. A width of the opening 651 is greater than the length of the injection device 10. A height of the opening 651 is greater than the width of the injection device 10. The opening 651 comprises a cutaway section 653 to enable the removal of an injection device 10 through the opening 651.

The packaging assembly 600 is configured to retain the injection devices 10 in a lateral arrangement, extending along the width of the case 610. The ejection mechanism is configured to push the injection devices 10 towards the opening 651. When an injection device 10 is removed through the opening 651, the ejection mechanism pushes the remaining injection devices 10 towards the opening 651. A spring of the ejection mechanism is arranged to push the injection devices 10 towards the opening 651. Alternatively, a flexible band may be provided, substantially as described with respect to the third embodiment.

The opening 651 forms a retaining lip to hold the injection devices 10 within the case 610. The lip is arranged along a lower side of the opening 651. The lip is arranged such that the ejection mechanism pushes the foremost injection device 10 against the lip. The injection device 10 can be removed from the case 610 by lifting the injection device 10 over the retaining lip. The cutaway section 653 is arranged such that the user can push on a lower side of the injection device 10 and lift the injection device 10 over the retaining lip. In this way, when the user pushes the injection device 10 upwards with their fingertips, the ejection mechanism pushes the injection device 10 out of the case 610 into the palm of the user. The injection devices 10 may easily be removed from the case 610 without closing the first or forming a pinch grip.

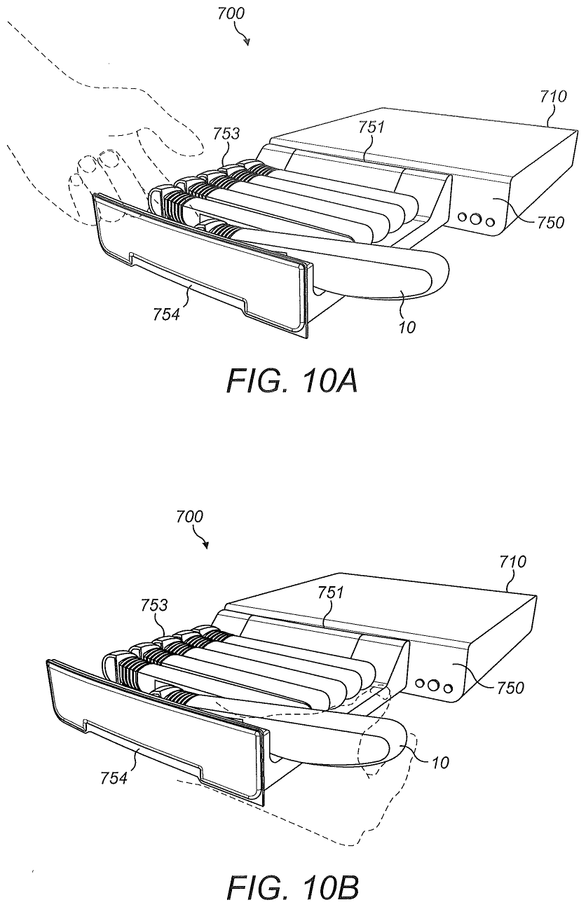

With respect to FIGS. 10A and 10B, a packaging assembly 700 according to a fourth embodiment is shown. Elements not described below are substantially the same as those of the first embodiment.

An internal structure 750 of the case 710 is formed with a single opening 751. The opening 751 is generally rectangular. The opening 751 extends over the full height of the case 710 and across the majority of the width of the case 710, up to the flat panel 753 which holds the user interface.

The case 710 comprises a tray 753 configured to hold the plurality of injection devices 10. The tray 753 is generally rectangular in shape. The tray 753 comprises a base, a front wall and a rear wall. The tray 753 is configured to slide into and out of the opening 751. A retaining element at the rear end of the tray 753 may prevent the tray 753 from being fully removed from the case 710. The front wall of the tray 753 is formed to have a handle 754 on an outward face, to pull the tray 753 out of the case 710. The handle 754 is formed by an opening or cutaway section of the outward face, with an overhanging lip or protrusion to be grasped.

The tray 753 is open on an upper side, such that the plurality of injection devices 10 can be dropped into and lifted out of the tray 753. The injection devices 10 are held in a lateral arrangement, extending along the width of the case 710. The base is formed to have an incline, descending from the rear end of the tray to the front end of the tray 753. In this way, the plurality of injection devices 10 can slide towards the front end of the tray 753. As shown in the figure, when the tray 753 is withdrawn from the case 710, the injection devices 10 may be pushed sideways out of the tray 753. The injection devices 10 may pushed with one hand until they fall into the other hand of the user. In this way, the injection devices 10 can be easily removed from the tray 753 without needing to form a pinch grip or a closed fist.

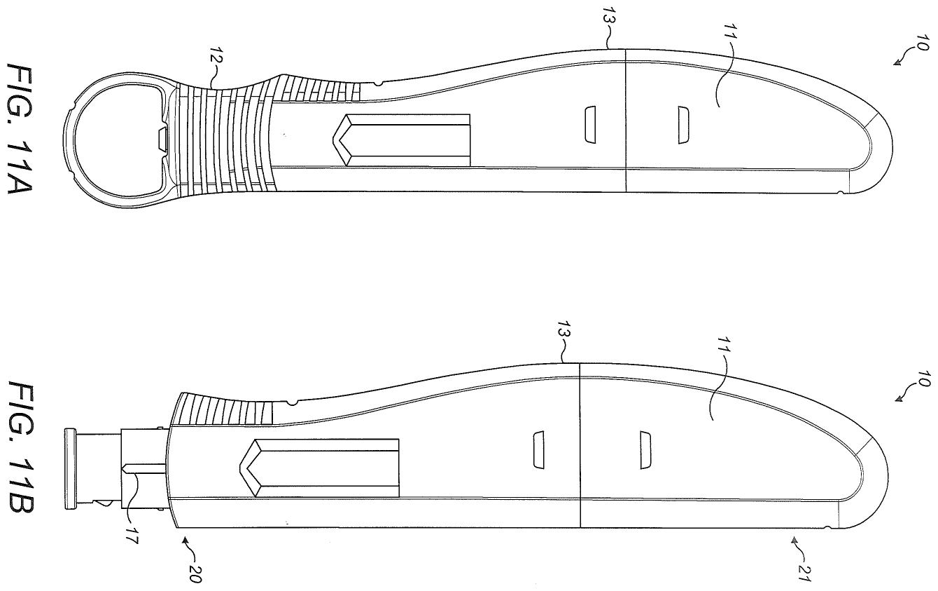

With respect to FIGS. 11A and 11B, an exemplary injection device 10 is shown. Injection device 10, as described above, is configured to inject a medicament into a user's body. Injection device 10 includes a housing 11 which typically contains a reservoir containing the medicament to be injected (e.g., a syringe) and the components required to facilitate one or more steps of the delivery process. As shown, housing 11 is formed in an ergonomic shape. The housing 11 is generally rectangular in shape, with one side face enlarged to form a curved outer surface 13. The curved outer surface 13 improves the fit of the housing in a user's hand, and allows the injection device 10 to be gripped without forming a closed first or a pinch grip. The edges of the housing 11 are generally rounded for user comfort when holding the injection device 10.

Injection device 10 can also include a cap assembly 12 that can be detachably mounted to the housing 11. Typically a user must remove cap 12 from housing 11 before injection device 10 can be operated. The cap 12 comprises a ring to pull the cap 12 from the housing 11. An internal diameter of the ring is large enough to pass a finger through the ring and pull the cap 12. In this way, it is possible to pull the cap 12 without forming a pinch grip. The enlarged curved outer surface 13 of the housing 11 can prevent the injection device 10 from sliding out of the user's hand when the cap 12 is pulled, even with only a loose grip.

The housing 11 has a distal region 20 and a proximal region 21. The term “distal” refers to a location that is relatively closer to a site of injection, and the term “proximal” refers to a location that is relatively further away from the injection site.

Insertion of needle 17 can occur via several mechanisms. For example, needle 17 may be fixedly located relative to housing 11 and initially be located within an extended needle sleeve. Proximal movement of the sleeve by placing a distal end of the sleeve against a user's body and moving housing 11 in a distal direction will uncover the distal end of needle 17. Such relative movement allows the distal end of needle 17 to extend into the user's body. Such insertion is termed “manual” insertion as needle 17 is manually inserted via the user's manual movement of housing 11 relative to the sleeve.

Another form of insertion is “automated,” whereby needle 17 moves relative to housing 11. Such insertion can be triggered by movement of the sleeve or by another form of activation, such as, for example, a button. A button may be located at a proximal end of housing 11 or, in other embodiments, a button could be located on a side of housing 11.

Other manual or automated features can include drug injection or needle retraction, or both. Injection is the process by which a bung or piston is moved from a proximal location within a syringe (not shown) to a more distal location within the syringe in order to force a medicament from the syringe through needle 17. In some embodiments, a drive spring (not shown) is under compression before device 10 is activated. A proximal end of the drive spring can be fixed within proximal region 21 of housing 11, and a distal end of the drive spring can be configured to apply a compressive force to a proximal surface of the piston. Following activation, at least part of the energy stored in the drive spring can be applied to the proximal surface of the piston. This compressive force can act on the piston to move it in a distal direction. Such distal movement acts to compress the liquid medicament within the syringe, forcing it out of needle 17.

Following injection, needle 17 can be retracted within the sleeve or housing 11. Retraction can occur when the sleeve moves distally as a user removes device 10 from a user's body. This can occur as needle 17 remains fixedly located relative to housing 11. Once a distal end of the sleeve has moved past a distal end of needle 17, and needle 17 is covered, the sleeve can be locked. Such locking can include locking any proximal movement of the sleeve relative to housing 11.

Another form of needle retraction can occur if needle 17 is moved relative to housing 11. Such movement can occur if the syringe within housing 11 is moved in a proximal direction relative to housing 11. This proximal movement can be achieved by using a retraction spring (not shown), located in distal region 20. A compressed retraction spring, when activated, can supply sufficient force to the syringe to move it in a proximal direction. Following sufficient retraction, any relative movement between needle 17 and housing 11 can be locked with a locking mechanism. In addition, a button or other components of device 10 can be locked as required.

It will be appreciated that the above described embodiments are purely illustrative and are not limiting on the scope of the claims. Other variations and modifications will be apparent to persons skilled in the art upon reading the present application, and some will now be described.

The case of the packaging arrangement may be a generally rectangular shape or may be any other shape suitable for containing the plurality of injectors. The case may be a suitable shape and size for placement within a household refrigerator.

The case may be formed to enclose the injectors and may be sealed. Alternatively, the case may be formed as a structure for supporting the plurality of injection devices externally. The injectors may be arranged in one or more rows, e.g. a row of six or two rows of three, or in a circular arrangement. The injectors may be arranged to hang below a supporting structure.

The case may be configured to store any number of injection devices, according to the dosage requirements of the medicament. For example, the case may store between 5 and 15 injection devices. Case may be sized to store enough injection devices for one quarter, or for a 6 month period. Where medicament is administered more regularly, the case may store enough injectors for one week.

The case may be formed of an opaque material. One or more of the components of the case may be formed with at least a transparent portion. A transparent portion of the case may allow the user to see the number of injection devices, or to see the user interface. One or more components of the case may be translucent to improve visibility of a visual reminder output.

The case may be formed of a plastics material such as polyethylene, polystyrene, polycarbonate, or it may be made of any other suitable material. Desired properties for the material of the case include temperature stability, moderate impact strength, resistance to cleaning fluids, a wipe-clean finish, and rigidity.

Each part of the case may be formed in a single piece e.g. a moulded plastic part. Alternatively, parts may be machined. The body of the case may be formed from two parts joined or attached together, or may be formed in a single part. An internal of the case may be formed as a single large cavity, a cavity divided into a plurality of areas for holding each injector, or may be formed as a plurality of cavities for individually holding each injector.

The case may comprise any number of magnets sufficient to support the weight of the packaging arrangement and injection devices. For example, the case may include 2 larger magnets or an arrangement of 6 smaller magnets. The magnets may be any permanent magnets and may be rare earth magnets. The magnets may be formed of neodymium or may be formed of samarium cobalt.

The case may further comprise one or more ventilating apertures to allow air flow into the case. Alternatively, the case may be sealed when the lid is in a closed position. The lid may further comprise a rubber seal to prevent air passing into the case between the lid and the case. The case may be insulated to maintain the low temperature of the injectors if removed from the fridge for a short period of time.

The lid may be coupled to the case with a hinge. The mechanism for connecting the lid to the case and for allowing the lid to open and close may take any suitable form. Instead of the hinge mechanism described above, the hinge may be a butt hinge, a living hinge or some other type. The lid may be coupled to the case with a flexible and/or elastic material. The hinge may allow some translational movement as well as pure rotational movement, to allow better viewing of or access to the internal part of the case when the lid is open.

The hinge may allow the removal of the lid by a user. For instance, the protrusions of each of the second hinging parts may be pushed inwards to disengage from the respective first hinging parts and decouple the lid from the case. The user may be provided with one or more alternative lids which may be a different design, for example, a different colour. An alternative lid may have a larger transparent portion or may be entirely opaque.

Alternatively, the lid may slidably engage with the case. The lid may comprise runners at the edges, each configured to engage with a corresponding groove on the case. The lid may slide out of the grooves and decouple from the case. The lid may be arranged to slide to the limit of the grooves and pivot freely in the open position. Further alternatively, the lid may be separate from the case and fixedly attached thereto with a friction fit. The lid may fit tightly within the opening at the front end of the case, or may fit over a front portion of the case.

The lid may comprise a latch to maintain the lid in the closed position. The latch may comprise a sliding catch arranged to slidably move between a first position and a second position. The catch may be arranged to protrude from an edge of the lid in the first position. The catch may be configured to slidably retract to not protrude in the second position. The latch may comprise a spring to urge the catch to the first position. The catch may be configured to engage with an opening in the case in the first position when the lid is in the closed position. The catch may engage with the opening to maintain the lid in the closed position.

The latch may be a sprung push-catch push-release mechanism. The latch may be configured to engage with a first push into the closed position and maintain the lid in the closed position. The latch may be configured to disengage with a second push and allow the lid to open. The latch may be configured to engage when the lid is closed to hold the lid in the closed position. The latch may further comprise a release switch to disengage the latch and allow the lid to open. The release switch may be a mechanical switch or an electric switch. The release switch may be an electric switch coupled to a code input, which is configured to disengage the lid catch when a correct code is entered.

Although the lid open sensor is described as an electromechanical switch, it may instead be an optical sensor arrangement, a magnetic sensor arrangement or any other suitable arrangement that is configured to detect whether the lid is open or closed or whether the lid is transitioning from a closed position to an open position.

The packaging assembly may comprise a case without a lid. The packaging assembly may not include a lid open sensor. The speaker may instead be deactivated by the processor arrangement according to an alert timer. The processor arrangement may be configured to operate the alert timer. The processor arrangement may activate the alert timer when the speaker is controlled to output an audio reminder alert that the scheduled dosing time is due. The processor arrangement may activate the alert timer when the scheduled dosing time is due, conditional on a fridge door being open. The processor arrangement may deactivate the speaker when the alert timer reaches 30 seconds. Alternatively, the processor arrangement may activate the alert timer at 20 seconds and count down until the timer expires. The processor arrangement may be configured to deactivate the speaker when the alert timer expires. The expiry time period for the alert timer may be 5 seconds to 60 seconds.

The electronics system may include an injector sensor to determine whether an injector is positioned in one of the plurality of openings. The injector sensor may determine whether an injector is positioned within each of the openings. The processor arrangement may be configured to deactivate the speaker when the injector sensor indicates that an injector has been removed from an opening.

The injector sensor may comprise one or more injector switches. The injector switches may be arranged respectively within the openings. Each injector switch may be a mechanical switch. The injector switch may be a normally open switch which is pressed to a closed position by an injector pen when in position in the opening. The injector switch may be a membrane switch. The injector switch may be actuated by a lever located within the opening.

Each injector switch may be configured to send a signal to the processor arrangement when an injector is located within the corresponding opening. The processor arrangement may be configured to deactivate the speaker when a signal is no longer received from an injector switch. The processor arrangement may be configured further to reset the countdown to the scheduled dosing time when an injector is removed from the opening. Alternatively, where an injector is replaced in the case after the dose is administered, the processor arrangement may be configured to reset the countdown when the injector is replaced. The processor arrangement may be configured to monitor the number of injectors in position in the packaging assembly. The processor arrangement may control the display to show the number of injectors in the packaging assembly. The processor arrangement may control the electronics system to provide a notification output when the packaging assembly is empty.

The retention mechanism may be arranged at the lower end of the case. The retention mechanism may be arranged to engage with the end of each injector which is passed through the opening. The retention mechanism may comprise a further plurality of openings at the lower end of the case. The further openings may be sized so as to hold the injectors in position with a friction fit. Alternatively, the retention mechanism may comprise a levered pincer arrangement arranged to grip the sides of an injector when the injector is pushed longitudinally into the arrangement, and to release the injector when the injector is pulled longitudinally out of the arrangement.

The retention mechanism may comprise a release switch configured to disengage the retention mechanism. The release switch may be configured to release one or all of the injectors. A plurality of release switches may be provided for the corresponding plurality of injectors. The release switch may be a mechanical switch or lever coupled to the retention mechanism. The release switch may be further coupled to the ejection mechanism. The release switch may be an electromechanical switch. The release switch may be controlled by the processor arrangement. The processor arrangement may control the release switch to disengage the retention mechanism conditional on the scheduled dosing time being due. The processor arrangement may control the release switch to disengage the retention mechanism for one injector when the scheduled dosing time is due. The retention mechanism may be controlled to release one injector, which is pushed partially out of the opening by the ejection mechanism. This arrangement may provide a visual reminder alert in the form of a portion of the injector being pushed out of the opening.

The band of the ejection mechanism may be formed from any suitably flexible material. For example, the band may be made of fabric, rubber, silicone or any other suitable material. The ring at the second end of the band may be formed from plastic or alternatively, from metal, rubber, wood or any other suitable material. The ring may be attached to or integrated with the band.

The ejection mechanism may comprise a motorised actuator. For example, a roller arranged perpendicularly to the plurality of injectors may be driven to push the injectors out of the openings. The roller may push all of the injector equally, with the retention mechanism configured to hold all but one of the injectors in position. Further alternatively, the actuator may comprise a protruding part from the rear of the case which is driven laterally across the width of the case. The protruding part may be driven along a rail, or may protrude from a belt extending along the width of the case. The protruding part is configured to engage with each injector in turn and push the injector out of the opening.

The time period for a reminder may be any suitable dosing period, dependent upon the medicament which is stored in the packaging assembly. The time period set until the next scheduled dosing time may be any number of days and may be, for example, between 2 and 60 days. The time period may be a number of weeks, for example, a period of 7 days, 14 days, 21 days or 28 days. The time period may be 28 days, which is 4 weeks, or the time period may be 1 month.

Alternatively, the time period may be 1 or 2 days, and the display may be configured to show the number of hours until the scheduled dosing time is due. Similarly, for a time period on the order of a number of hours, the display may show a number of minutes.

The timer duration switch may be configured to select between any two time periods. For example, the first switch position may correspond to a time period of 7 days and the second switch position may correspond to a time period of 14 days. Alternatively, the timer duration switch may be a multi-positional switch, for example, a rotary switch or a dial. The time period may be set in conjunction with the display, wherein a first user input causes the display to show the current time period, and a second input is used to adjust the time period.

The reset timer may operate on any suitable timeframe. For example, the reset button may be configured to reset the countdown timer if pressed for 1 second or up to 5 seconds.

The display may comprise more than 2 LED arrays, to accommodate larger numbers and messages, or may be a single LED array only. Alternatively, the display may comprise any form of electronic display suitable for displaying a number and/or a message, for example, the display may be an array of LED pixels, an LCD or e-paper screen, or a split-flap display. The display may be arranged in a peripheral module which is separate from the case. The display module may be connected to electrics system with a wired or wireless connection. The electronics system may comprise any display driver which is suitable for chosen display.

The display may be configured to provide further status information, or more detail, in the form of text messages on the display. For example, the display may provide a visual reminder that the scheduled dosing time is due by showing a reminder message in addition to, or instead of, flashing the number 00. The output of the number 00 is an example of a reminder message. The display may be controlled to show the number of injectors remaining in the packaging assembly. The processor arrangement may be configured to determine the number of injectors according to an input from an injector sensor. Alternatively, the processor arrangement may be configured to monitor the number of times that a scheduled dosing time has passed. The display may be controlled to show a notification message when the packaging assembly is empty.

The processor arrangement may be configured to deactivate the display of the user interface if the lid of the case is closed. The processor arrangement may activate the LED based on the fridge open sensor, to indicate to the user that the status of the packaging assembly is normal, when the number of days remaining is greater than one, whether the lid is closed or not. The user may open the lid to activate the display and show the number of days if required. When the scheduled dosing time is due, the processor arrangement may activate both the LED and the display to flash, in order to provide a visual reminder, whether the lid is open or not.

More than one LED may be included in the user interface to indicate the status of the packaging assembly in more detail. For example, a status LED having a first colour may be activated when the number of days until the scheduled dosing time is greater than one, the status LED may flash when the number of days remaining is one day, and a second LED having a second colour may be activated when the scheduled dosing time is due. Alternatively, a single two-colour LED may be used. Alternatively, any other form of notification light or visual output transducer may be used in place of the LED.

A plurality of LEDs corresponding to the plurality of openings may be provided with the packaging assembly of any embodiment. The LEDs may be controlled as described with respect to the fourth embodiment. Alternatively, the processor arrangement may flash or blink one of the plurality of LEDs, while the remaining LEDs are off or illuminated continuously. A different LED may be controlled to blink each time, to guide the user to the next injector for use. One LED may be flashed in a different colour. The processor arrangement may control a number of LEDs according to the number of injection devices remaining in the packaging assembly.

The speaker may be any suitable form of audio output transducer, for example, an electro-acoustic transducer, a piezoelectric buzzer, a moving diaphragm speaker, or a mechanical bell. A vibrating alert may be used instead of or in addition to the audio output transducer.

A fridge open sensor may be included comprising a phototransistor or, alternatively, a photoresistor or photodiode. Alternatively, the fridge open sensor may comprise a mechanical switch. The fridge open sensor may be located externally from the case and may be positioned at a hinge or frame of the fridge door. The fridge open sensor may be a mechanical switch which is arranged to be pressed by the fridge door in a closed position.

A door open timer may be operated, for example, the user interface may enter a partial sleep mode if the fridge door is open for 10 minutes or 15 minutes.

Alternative countdown timer implementations include off-chip and on-chip state-based logic circuits with clock devices, and other forms will be apparent to the skilled person.

The PCB and components of the electronics system may be sealed for protection. For example, the PCB may be coated on each side with a water resistant lacquer or another suitable coating. The electronics system may be coated for protection from moisture or humidity in the interior of a household fridge.

The packaging assembly may include a greater or smaller number of batteries, according to the power requirements of the electronics system. For example, the packaging assembly may include a single battery power pack. The battery or batteries may be removable and replaceable, or may be fixed within the case of the packaging assembly. Alternatively, the packaging assembly may be adapted for a mains power supply, or any alternative power supply.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The term “drug delivery device” shall encompass any type of device or system configured to dispense a drug or medicament into a human or animal body. Without limitation, a drug delivery device may be an injection device (e.g., syringe, pen injector, auto injector, or other device configured for intraocular, subcutaneous, intramuscular, or intravascular delivery), skin patch (e.g., osmotic, chemical, micro-needle), inhaler (e.g., nasal or pulmonary). The presently described drugs may be particularly useful with injection devices that include a needle, e.g., a hypodermic needle for example having a Gauge number of 24 or higher.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to any substance which is sufficiently structurally similar to the original substance so as to have substantially similar functionality or activity (e.g., therapeutic effectiveness). In particular, the term “analogue” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyhepta-′decanoyl) human insulin.

Examples of GLP-1, GLP-1 analogues and GLP-1 receptor agonists are, for example, Lixisenatide (Lyxumia®, Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An example of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

1. A packaging assembly comprising:
a case configured to at least partially contain a plurality of injection devices for delivering a medicament, the case being formed as a box having at least one open end face;
a lid coupled to the case and movable between an open position and a closed position, the lid being configured to hinge at an edge of the lid furthest from the at least one open end face of the case on another first face of the case, and being arranged in the closed position to extend substantially over the open end face of the case and at least partially over another second face of the case that is adjacent to the open end face; and
a handle portion having an upper side and a lower side and being arranged to protrude from the lid such that pressure applied to the upper side when the lid is in the closed position causes the lid to move to the open position, and pressure applied to the lower side when the lid is in the open position causes the lid to move to the closed position.

2. The packaging assembly of claim 1, wherein the handle portion is formed to extend longitudinally along the lid in parallel with a boundary formed between a first portion of the lid extending over the open end face of the case and a second portion of the lid extending partially over the other first face of the case.

3. The packaging assembly of claim 1, further comprising one or more spring ejectors configured to urge at least one injection device of the plurality of injection devices at least partially out of the case when the lid is in the open position.

4. The packaging assembly of claim 3,
wherein the case is configured to contain the plurality of injection devices in an arrangement wherein each injection device of the plurality of injection devices lies perpendicular to the open end face of the case, and
wherein the case comprises one spring ejector for each injection device of the plurality of injections devices, each spring ejector being configured to urge at least a portion of a corresponding injection device of the plurality of injection devices through the open end face of the case.

5. The packaging assembly of claim 3,
wherein the case is configured to contain the plurality of injection devices in an arrangement wherein each injection device of the plurality of injection devices lies parallel to the open end face of the case, and
wherein the case comprises one spring ejector configured to urge the plurality of injection devices toward the open end face of the case.

6. The packaging assembly of claim 5, further comprising a retaining lip and a release opening,
wherein the plurality of injection devices are urged against the retaining lip by the one spring ejector,
wherein the retaining lip is arranged to retain the plurality of injection devices within the case; and
wherein a release slot is arranged such that, through the release opening, one injection device of the plurality of injection devices can be lifted over the retaining lip.

7. The packaging assembly of claim 5, wherein the lid comprises a dispense slot configured to contain one injection device of the plurality of injection devices; and
wherein the one spring ejector is configured to urge the plurality of injection devices towards the lid, such that when the lid is moved from the closed position to the open position, one injection device of the plurality of injection devices is contained in the dispense slot.

8. The packaging assembly of claim 1,
wherein the case is configured to contain the plurality of injection devices in an arrangement wherein each injection device of the plurality of injection devices lies perpendicular to the open end face of the case, and
wherein the packing assembly further comprises a plurality of flexible bands, each flexible band of the plurality of flexible bands arranged to extend around an end of a corresponding injection device of the plurality of injection devices that is furthest from the open end face of the case, such that the corresponding injection device is urged towards the open end face of the case by the flexible band when the flexible band is pulled tight.

9. The packaging assembly claim 1, further comprising the plurality of injection devices.

10. The packaging assembly of claim 9, wherein each injection device of the plurality of injection devices comprises a loop element formed at one end of the injection device, and the case is configured to contain the plurality of injection devices in an arrangement wherein the loop element of each injection device is adjacent to the open end face of the case.

11. A packaging assembly comprising:
a case configured to at least partially contain a plurality of injection devices for delivering a medicament, the case formed as a box having at least one opening; and
at least one tray configured to contain at least one injection device of the plurality of injection devices and arranged to slide out of the case through the opening.

12. The packaging assembly of claim 11, further comprising one tray for each injection device of the plurality of injection devices.

13. The packaging assembly of claim 12, where each tray of the plurality of trays is configured to rotate around its length such that the injection device contained in the tray falls out of the tray.

14. The packaging assembly of claim 11, further comprising one tray configured to contain all of the plurality of injection devices.

15. The packaging assembly of claim 14, wherein the one tray is configured, when pulled fully out of the case, to hinge around an edge of the one tray that is adjacent to the case.

16. The packaging assembly of claim 14,
wherein the one tray is configured to contain the plurality of injection devices in an arrangement wherein each injection device of the plurality of injection devices lies perpendicular to a direction in which the one tray is configured to slide, and
wherein a side panel of the one tray comprises at least one opening for pushing one or more of the injection devices of the plurality of injection devices out of the one tray.

17. The packaging assembly of claim 16, wherein the one tray is formed having at least one sloped portion arranged to lift one or more of the injections devices of the plurality of injection devices out of the one tray when pushed through the at least one opening in the side panel.

18. The packaging assembly of claim 14,
wherein the one tray is configured to contain the plurality of injection devices in an arrangement wherein each injection device of the plurality of injection devices lies parallel to a direction in which the one tray is configured to slide.

19. The packaging assembly of claim 18, wherein a bottom panel of the one tray comprises at least one opening for pushing one or more injection devices of the plurality of injection devices upwards out of the one tray.

20. The packaging assembly of claim 11, further comprising the plurality of injection devices.
